(19) United States
(12) Patent Application Publication
(10) Pub. No.: US 2006/0128681 A1
(43) Pub. Date: Jun. 15, 2006

(54) Novel rho inhibitors

(76) Inventors: Harold Kohn, Chapel Hill, NC (US); Thomas P Weber, Arlington, VA (US); William R Widger, Stafford, TX (US)

Correspondence Address: MYERS BIGEL SIBLEY & SAJOVEC, PO BOX 37428, RALEIGH, NC, 27627, US

(21) Appl. No.: 10/520,778

(22) PCT Filed: Jun. 30, 2003
(86) PCT No.: PCT/US03/20491

§ 371 Date: Oct. 31, 2005

Related U.S. Application Data
(60) Provisional application No. 60/394,730, filed on Jul. 10, 2002.

(51) Int. Cl. A61K 31/555; A61K 31/28; A61K 31/315; A61K 31/295

(52) U.S. Cl. 514/184; 514/492; 514/494; 514/501; 514/505

(57) ABSTRACT

Rho is an enzyme essential for the growth and survival of Escherichia coli, and bicyclomycin is its only known selective inhibitor. The site and pathway of rho inhibition by the putative metal thiol chelates are disclosed. Additionally compositions and methods of divalent metals and compounds containing a thiol group are disclosed.

### CROSS REFERENCE TO RELATED APPLICATION

The present application claims priority to U.S. Provisional Application No. 60/394,730, filed Jul. 10, 2002, the disclosure of which is incorporated herein by reference in its entirety.

### STATEMENT OF FEDERAL SUPPORT

This invention was made with support from the United States Federal Government under grant numbers GM37934 from the National Institutes of Health. The United States Government has certain rights in this invention.

### FIELD OF THE INVENTION

The present invention relates to compositions and methods for treating a bacterial and/or fungal infection. More specifically the present invention relates to compositions comprising divalent metals and a compound containing a thiol group.

### BACKGROUND OF THE INVENTION

Rho-dependent transcription termination is a regulatory process that controls the expression of genes in most prokaryotes. Yager et al., (1987) In The Molecular and Cell Biology of E. coli and S. typhimurium, (Neidhardt, F., Ed.) Am. Soc. Microbiol., pp. 1241-1275, Washington, D.C. In Escherichia coli, select termination events require the presence of rho, and the disruption of rho function leads to loss of cellular viability. Richardson, (2002) Rho-dependent termination and ATPases in transcript termination, Biochim. Biophys. Acta 1577, 251-260.

Rho is a hexamer of 47-kDa identical subunits arranged in a toroidal shape with either C6/C3 symmetry or C6 in the absence of ATP. See, Richardson, (2002); and Vincent et al., (2000) Rho transcription factor: Symmetry and binding of bicyclomycin, Biochemistry 39, 9077-9083; and Yi et al., (2003) ATP binding to rho transcription termination factor. Mutant F355W ATP-induced fluorescence quenching reveals dynamic ATP binding, J. Biol. Chem., 278, 13719-13727. Mechanistic investigations support a “tethered-tracking” pathway for rho function in which rho binds to specific RNA sequences and then translocates (5′→3′) along the nascent RNA, using binding sites located within the central hole in a process fueled by Mg²⁺-mediated ATP hydrolysis. Richardson, (2002); and Xu et al., (2002) Mutations in the rho transcription termination factor that affect RNA tracking, J. Biol. Chem. 277, 30023-30030. Transcript termination results when rho encounters the stalled RNA polymerase.

Rho is unique to most prokaroytic bacteria and thus is an attractive macromolecular target for drug intervention. The only reported selective inhibitor of rho is the natural product and commercial agent bicyclomycin (1). Zwiefka et al., (1993) Transcription termination factor rho: The site of bicyclomycin inhibition in Escherichia coli, Biochemistry 32, 3564-3570.

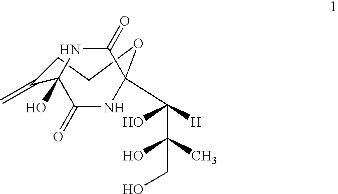

Bicyclomycin (1)

It has been demonstrated that bicyclomycin both interferes with rho RNA tracking and disrupts ATP hydrolysis. Magyar et al., (1996) The antibiotic bicyclomycin affects the secondary RNA binding site of Escherichia coli transcription termination factor Rho, J. Biol. Chem. 271, 25369-25374, see also, Weber et al., (2002) The Mg²⁺ requirements for rho transcription termination factor: Catalysis and bicyclomycin inhibition, Biochemistry 41, 12377-12383. However, bicyclomycin only affects one pathway of the transcription termination factor Rho. It may therefore be useful to find additional compositions that affect the pathway of the transcription termination factor Rho.

### SUMMARY OF THE INVENTION

The present invention discloses pharmaceutical composition comprising divalent metals and compounds comprising at least one thiol group, or a pharmaceutically acceptable salt or prodrug thereof, wherein said divalent metal and said at least one thiol group form a metal:thiol complex, in association with a pharmaceutically acceptable carrier, excipient or diluent.

The present invention further discloses methods for inhibiting rho transcription termination factors, wherein the methods include administering a chelating agent and a compound comprising at least one thiol group.

The present invention also discloses methods of treating microorganisms by administering a therapeutically effective amount of a composition including a divalent metal and a compound comprising at least one thiol group. Furthermore, the present invention discloses methods of treating bacteria and fungi through the administration of a therapeutically effective amount of a composition. The present invention may be used for treating both gram-negative and gram-positive bacteria.

The foregoing and other aspects of the present invention are explained in detail in the specification set forth below.

### BRIEF DESCRIPTION OF THE DRAWINGS

FIG. 1 is a graph depicting metal-(DL)DTT(1:1) inhibition of rho poly(C)-dependent ATPase activity. The reactions were conducted using a 100 μL-solution containing ATPase buffer, rho (250 nM (monomer)), poly(C) (250 nM), ATP (200 μM), MgCl₂ (1 mM), and metal-(DL)DTT (1:1) (0-500 μM) at 32° C. The average velocities of two determinations are plotted. Zn-(DL)DTT (♦); Cd-(DL)DTT (▪); Ni-(DL)DTT (▾); Mn-(DL)DTT (χ).

FIG. 2A illustrates the spectrum activity of Zn-(L)DTT inhibition of rho poly(C)-dependent ATPase activity as a function of ZnCl₂ and (L)DTT concentration. The reactions were conducted using a 100 μL-solution containing ATPase buffer, rho (200 nM (monomer)), poly(C) (200 nM), ATP (200 μM), MgCl₂ (10 mM), and combinations of ZnCl₂ (0-320 μM) and (L)DTT (0-320 μM) at 32° C. The average normalized velocities (z-axis) of two determinations are plotted as a function of total ZnCl₂ (y-axis) and total (L)DTT concentration (x-axis).

FIG. 2B is a graph depicting Zn-(L)DTT inhibition isobals. The isobals (20, 40, 60, 80, 100%) give the degree of enzyme activity. The shaded lines correspond to 1:2, 1:1, and 2:1 ZnCl₂ to (L)DTT solutions, respectively.

FIG. 3A illustrates Zn-(L)DTT inhibition of rho in the poly(C)-dependent ATPase assay. The double, reciprocal plot of rho poly(C)-dependent ATPase activity versus ATP concentrations for varying concentrations of Zn-(L)DTT (1:1). Values listed are the average of duplicate reactions. The inset illustrates a plot of the slopes (♦) and intercepts (▪) versus Zn-(L)DTT concentrations.

FIG. 3B depicts a graph of Zn-(L)DTT inhibition of rho in the poly(dC)-ribo(C)₁₀-dependent ATPase assay. The double, reciprocal plot of the poly(dC)-ribo(C)₁₀-dependent ATPase activity of rho with varying concentrations of Zn-(L)DTT (1:1). Values listed are the average of duplicate reactions. The inset shows a plot of the slopes (♦) and intercepts (▪) versus Zn-(L)DTT concentrations.

FIG. 4 is a promiscuity test for Zn-(L)DTT. The reactions were conducted using a 100 μL-solution containing rho (100 nM (monomer)), poly(C) (100 nM), ATP (200 μM), MgCl₂ (10 mM) and Zn-(L)DTT (0-800 μM) at 32° C. The average velocities of two determinations are plotted. Control (♦); no preincubation (▪); 10-fold concentration of rho, poly(C) and ATP (▴); standard condition +0.1 mg/mL BSA (χ).

FIG. 5 shows the effect of preincubation time on Zn-(L)DTT inhibition. The reactions were conducted using a 100 μL-solution containing rho (100 nM (monomer)), poly(C) (100 nM), ATP (200 μM), MgCl₂ (10 mM) and Zn-(L)DTT (0-400 μM) at 32° C. The average velocities of two determinations are plotted. No preincubation (♦); 15 sec (▪); 30 sec (▴); 1 min (χ); 2 min (χ); 5 min (●).

FIG. 6 is a promiscuity test for bicyclomycin. The reactions were conducted using a 100 μL-solution containing rho (100 nM (monomer)), poly(C) (100 nM), ATP (200 μM), MgCl₂ (10 mM) and bicyclomycin (0-400 μM) at 32° C. The average velocities of two determinations are plotted. Control (♦); No preincubation (▪); 10-fold concentration of rho, poly(C) and ATP (▴); standard condition +0.1 mg/mL BSA (χ).

FIG. 7 illustrates the structure-activity relationship for a series of Zn²⁺ chelates (1:1). The reactions were conducted using a 100 μL-solution containing rho (100 nM (monomer)), poly(C) (100 nM), ATP (200 μM), MgCl₂ (10 mM) and Zn-chelate (0-400 μM) at 32° C. The average velocities of two determinations are plotted. Zn-2-mercaptoethanol (♦); Zn-1,2-ethanedithiol (χ); Zn-(L)DTT (●).

FIG. 8 illustrates metal activation of rho exhibiting a peak velocity. The reactions were conducted using a solution (100 μL) containing ATPase buffer, rho (100 nM (monomer)), poly(C) (100 nM), ATP (200 μM), and various concentrations of total M²⁺ at 32° C. The average velocities of two determinations are plotted with Mg²⁺ (diamonds), Mn²⁺ (squares), Zn²⁺ (gray triangles), and Cd²⁺ (×). The inset shows metal activation of rho exhibiting a peak velocity and sigmoidal behavior at rho divalent metal concentrations.

### DETAILED DESCRIPTION OF THE PREFERRED EMBODIMENTS

The present invention will now be described more fully hereinafter with reference to the accompanying figures, in which preferred embodiments of the invention are illustrated. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

Some of the embodiments of the present invention relate to pharmaceutical compositions comprising a divalent metal; and a compound comprising at least one thiol group, or a pharmaceutically acceptable salt or prodrug thereof, wherein said divalent metal and said at least one thiol group form a complex comprising a metal chelated by a thiol compound in the form a metal:thiol complex, in association with a pharmaceutically acceptable carrier, excipient or diluent. The compound may be an optical isomer, enantiomer, diastereomer, racemate or racemic mixture thereof of the compound.

The term “thiol” is used herein to refer to a compound that contains one or more sulfur atoms capable of existing in the form of sulfhydryl groups under appropriate pH conditions, e.g. significantly below the lowest pKa of the compound, regardless of whether such sulfur atoms are deprotonated or fully or partially protonated under conditions in which the thiol is used. “Thiol group” means a sulfur or an —SH group of a “thiol”. Thus, the sulfur may be a free radical or a protonated species

The compound comprising at least one thiol group may comprise a cyclic or acyclic carbon framework. The cyclic or acyclic carbon framework may comprise moieties of 2-10 carbons that may be linear or branched. Furthermore, the cyclic or acyclic carbon framework may comprise either electron-donating or electron-withdrawing groups. Additionally, the cyclic or acyclic carbon framework may be aliphatic, heterocyclic, aromatic, or heteroaromatic. Furthermore, the cyclic or acyclic carbon framework may include single, double or triple bonds. The compounds used may be optically pure, racemic, or mixed optical compositions.

The divalent metal may include Ba²⁺, Be²⁺, Ca²⁺, Cd²⁺, Co²⁺, Cu²⁺, Fe²⁺, Hg²⁺, Mn²⁺, Ni²⁺, Pd²⁺, Pt²⁺, Sn²⁺, Sr²⁺, and Zn²⁺. Furthermore, a metal oxide may be utilized such as VO²⁺ or TiO₂. The divalent metal or metal oxide may include any light metal or transition metal resulting in a divalent cation.

The compound comprising at least one thiol group may include dithiothreitol, dithioerythritol, 1,4-dihydroxy-2,3-dimercaptobutane, 1,2-ethanedithiol, 3,4-dimercaptotoluene, trithiocyanuric acid, 2,5-dimercapto-1,3,4-thiadiazole, 2,3-dimercapto-1-propanol, 2-mercapto-3-butanol, β-mercaptoethanol, 2-mercaptoethylamine, 1-monothioglycerol, 2,3-butanedithiol, 1,4-butanedithiol, 1,2-propanedithiol, 1,3-propanedithiol, benzene-1,2-dithiol, 1,2-benzenedimethanedithiol, 2,3-dimercaptopyridine, 3,4-dimercaptopyridine, 4-mercaptopyridine, 3-mercaptopyridine, 2-mercaptopyridine, 2-mercaptoethylsulfide, 2-mercaptoethyl ether, and bis-(2-mercaptoethyl)amine. These compounds may act as chelating agents. Other chelating agents may include benzenedithiol, mercaptoacetic acid, thiosalicyclic acid, 2-mercaptopyridine-N-oxide and dimethyldithiocarbamic acid. The preceding compounds can be substituted with either electron-donating or electron-withdrawing groups, and where appropriate can be used as optically pure, racemic, or mixed optical compositions.

A “therapeutically effective” amount as used herein is an amount of the composition disclosed above to treat bacterial and/or fungal infections in a subject. The therapeutically effective amount will vary with the age and physical condition of the patient, the severity of the treatment, the duration of the treatment, the nature of any concurrent treatment, the pharmaceutically acceptable carrier used and like factors within the knowledge and expertise of those skilled in the art. Pharmaceutically acceptable carriers are preferably solid dosage forms such as tablets or capsules. Liquid preparations for oral administration may be also be used and may be prepared in the form of syrups or suspensions, e.g., solutions containing an active ingredient, sugar, and a mixture of ethanol, water, glycerol, and propylene glycol. If desired, such liquid preparations may contain coloring agents, flavoring agents, and saccharin. Thickening agents such as carboxymethylcellulose may also be used. Additionally, transdermal patches and other acceptable carriers, the selection of which are known in the art.

Some of the embodiments of the present invention also contemplate pharmaceutical formulations, both for veterinary and for human medical use, which comprise as the active pharmaceutical ingredient one or more compound(s) of the present invention. In such pharmaceutical and medicament formulations, the active pharmaceutical ingredient preferably is utilized together with one or more pharmaceutically acceptable carrier(s) therefor and optionally any other therapeutic ingredients. The carrier(s) must be pharmaceutically acceptable in the sense of being compatible with the other ingredients of the formulation and are preferably not unduly deleterious to the recipient thereof. The active pharmaceutical ingredient is provided in an amount effective to achieve the desired pharmacological effect, as described above, and in a quantity appropriate to achieve the desired daily dose.

The formulations include those suitable for parenteral as well as non-parenteral administration, and specific administration modalities include, but are not limited to, oral, rectal, buccal, topical, nasal, ophthalmic, subcutaneous, intramuscular, intravenous, transdermal, intrathecal, intra-articular, intra-arterial, sub-arachnoid, bronchial, lymphatic, vaginal, and intra-uterine administration. Formulations suitable for oral and parenteral administration are preferred, with formulations suitable for oral administration most preferred.

When a compound of the present invention is utilized in a formulation comprising a liquid solution, the formulation advantageously may be administered orally or parenterally. When a compound of the present invention is employed in a liquid suspension formulation or as a powder in a biocompatible carrier formulation, the formulation may be advantageously administered orally, rectally, or bronchially.

When a compound of the present invention is utilized directly in the form of a powdered solid, the compound may advantageously be administered orally. Alternatively, it may be administered bronchially, via nebulization of the powder in a carrier gas, to form a gaseous dispersion of the powder that is inspired by the patient from a breathing circuit comprising a suitable nebulizer device.

The formulations comprising a compound of the present invention may conveniently be presented in unit dosage forms and may be prepared by any of the methods well known in the art of pharmacy. Such methods generally include the step of bringing a compound of the present invention into association with a carrier that constitutes one or more accessory ingredients. Typically, the formulations are prepared by uniformly and intimately bringing a compound of the present invention into association with a liquid carrier, a finely divided solid carrier, or both, and then, if necessary, shaping the product into dosage forms of the desired formulation.

Formulations of the present invention suitable for oral administration may be presented as discrete units such as capsules, cachets, tablets, or lozenges, each containing a predetermined amount of a compound of the present invention as a powder or granules; or a suspension in an aqueous liquor or a non-aqueous liquid, such as a syrup, an elixir, an emulsion, or a draught.

A tablet may be made by compression or molding, optionally with one or more accessory ingredients. Compressed tablets may be prepared by compressing in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered active compound with a suitable carrier may be made by molding in a suitable machine.

A syrup may be made by adding a compound of the present invention to a concentrated aqueous solution of a sugar, for example sucrose, to which may also be added any accessory ingredient(s). Such accessory ingredient(s) may include, for example, flavorings, suitable preservatives, agents to retard crystallization of the sugar, and agents to increase the solubility of any other ingredient, such as a polyhydroxy alcohol, for example glycerol or sorbitol.

Formulations suitable for parenteral administration conveniently comprise a sterile aqueous preparation of a compound of the present invention, which preferably is isotonic with the blood of the recipient (e.g., physiological saline solution). Such formulations may include suspending agents and thickening agents or other microparticulate systems which are designed to target the compound to blood components or one or more organs. The formulations may be presented in unit-dose or multi-dose form.

Nasal spray formulations comprise purified aqueous solutions of a compound of the present invention with preservative agents and isotonic agents. Such formulations are preferably adjusted to a pH and isotonic state compatible with the nasal mucus membranes.

Formulations for rectal administration may be presented as a suppository with a suitable carrier such as cocoa butter, hydrogenated fats, or hydrogenated fatty carboxylic acid.

Ophthalmic formulations are prepared by a similar method to the nasal spray, except that the pH and isotonic factors are preferably adjusted to match that of the eye.

Topical formulations comprise a compound of the present invention dissolved or suspended in one or more media, such as mineral oil, petroleum, polyhydroxy alcohols, or other bases used for topical pharmaceutical formulations. In addition to the aforementioned ingredients, the formulations of this invention may further include one or more accessory ingredient(s) selected from diluents, buffers, flavoring agents, disintegrants, surface active agents, thickeners, lubricants, preservatives (including antioxidants), and the like.

In some embodiments, the drug product may be present in a solid pharmaceutical composition that may be suitable for oral administration. A solid composition of matter according to the present invention may be formed and may be mixed with and/or diluted by an excipient. The solid composition of matter may also be enclosed within a carrier which may be, for example, in the form of a capsule, sachet, tablet, paper, or other container. When the excipient serves as a diluent, it may be a solid, semi-solid, or liquid material which acts as a vehicle, carrier, or medium for the composition of matter.

Various suitable excipients will be understood by those skilled in the art and may be found in the National Formulary, 19: 2404-2406 (2000), the disclosure of pages 2404 to 2406 being incorporated by reference herein in their entirety. Examples of suitable excipients include, but are not limited to, starches, gum arabic, calcium silicate, microcrystalline cellulose, methacrylates, shellac, polyvinylpyrrolidone, cellulose, water, syrup, and methylcellulose. The drug product formulations can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to, mannitol, sorbitol, xylitol, sucrose, maltose, glucose, lactose, dextrose, and the like. Suitable buffers encompass, but are not limited to, phosphate, citrate, tartarate, succinate, and the like. Other inert fillers which may be used encompass those which are known in the art and are useful in the manufacture of various dosage forms. If desired, the solid formulations may include other components such as bulking agents and/or granulating agents, and the like. The drug products of the invention may be formulated so as to provide quick, sustained, or delayed release of the active ingredient after administration to the patient by employing procedures well known in the art.

To form tablets for oral administration, the composition of matter of the present invention may be made by a direct compression process. In this process, the active drug ingredients may be mixed with a solid, pulverant carrier such as, for example, lactose, saccharose, sorbitol, mannitol, starch, amylopectin, cellulose derivatives or gelatin, and mixtures thereof, as well as with an antifriction agent such as, for example, magnesium stearate, calcium stearate, and polyethylene glycol waxes. The mixture may then be pressed into tablets using a machine with the appropriate punches and dies to obtain the desired tablet size. The operating parameters of the machine may be selected by the skilled artisan. Alternatively, tablets for oral administration may be formed by a wet granulation process. Active drug ingredients may be mixed with excipients and/or diluents. The solid substances may be ground or sieved to a desired particle size. A binding agent may be added to the drug. The binding agent may be suspended and homogenized in a suitable solvent. The active ingredient and auxiliary agents may also be mixed with the binding agent solution. The resulting dry mixture is moistened with the solution uniformly. The moistening typically causes the particles to aggregate slightly, and the resulting mass is pressed through a stainless steel sieve having a desired size. The mixture is then dried in controlled drying units for the determined length of time necessary to achieve a desired particle size and consistency. The granules of the dried mixture are sieved to remove any powder. To this mixture, disintegrating, antifriction, and/or anti-adhesive agents are added. Finally, the mixture is pressed into tablets using a machine with the appropriate punches and dies to obtain the desired tablet size. The operating parameters of the machine may be selected by the skilled artisan.

If coated tablets are desired, the above prepared core may be coated with a concentrated solution of sugar or cellulosic polymers, which may contain gum arabic, gelatin, talc, titanium dioxide, or with a lacquer dissolved in a volatile organic solvent or a mixture of solvents. To this coating various dyes may be added in order to distinguish among tablets with different active compounds or with different amounts of the active compound present. In a particular embodiment, the active ingredient may be present in a core surrounded by one or more layers including enteric coating layers.

Soft gelatin capsules may be prepared in which capsules contain a mixture of the active ingredient and vegetable oil. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, and/or gelatin.

Other methods may utilize an aqueous medium which contains an active ingredient or ingredients, a quantity of one or more surfactants sufficient to dissolve or suspend said active ingredients uniformly throughout the medium and other manufacturing additives as known to the art. The latter include granulating-binding agents such as gelatin; natural gums, such as acacia, tragacanth; starches, sodium alginate, sugars, polyvinylpyrrolidone; cellulose derivatives such as hydroxypropylmethylcellulose, polyvinyloxoazolidones; pharmaceutical fillers such as lactose, microcrystalline cellulose, dicalcium phosphate, tricalcium phosphate, calcium sulfate, dextrose, mannitol, sucrose; tabletting lubricants if needed such as calcium and magnesium stearate, stearic acid, talc, sterotex (alkaline stearate).

The components may then be granulated, the resulting granules are dried, sieved and compressed into tablets or filled into capsules. Other oral product forms may be similarly prepared by art methods such as chewable tablets, lozenges, troches, sustained or delayed release products or suspensions.

In the event that the above pharmaceuticals are to be used for parenteral administration, such a formulation may comprise sterile aqueous injection solutions, non-aqueous injection solutions, or both, comprising the composition of matter of the present invention. When aqueous injection solutions are prepared, the composition of matter may be present as a water soluble pharmaceutically acceptable salt. Parenteral preparations may contain anti-oxidants, buffers, bacteriostats, and solutes which render the formulation isotonic with the blood of the intended recipient. Aqueous and non-aqueous sterile suspensions may comprise suspending agents and thickening agents. The formulations may be presented in unit-dose or multi-dose containers, for example sealed ampules and vials. Extemporaneous injection solutions and suspensions may be prepared from sterile powders, granules and tablets of the kind previously described.

Accordingly, compounds according to the present invention may be utilized to treat microorganisms. The term “antimicrobial”, as used herein, refers to the ability to slow, reduce, terminate or inhibit the growth of microorganisms. Microorganisms which may be treated with compounds of the present invention include, but are not limited to, fungi, parasites, bacteria, viruses, etc. These compounds may be produced in antibiotics or as antiperspirants, skin sanitizers, dental rinses or any areas where microorganisms may grow. Additional applications include laundry detergents, fabric softeners, fabric softener sheets, (automatic) dishwasher detergents and all purpose cleaners. Further applications are air fresheners and odorants, odor masking agents and/or antimicrobial agents.

The term “treating” as used herein refers to the ability to slow, control, prevent, reduce, terminate or inhibit the growth of microorganisms.

For the purposes of this invention, the terms “inhibition of virus” and “virucidal activity” as used herein refer to a reduction in the amount of virus present in a sample contacted with the metal-containing compounds of this invention. In one embodiment, the terms refer to at least a 50% reduction in the amount of virus detected and preferably at least a 75% reduction in the amount of virus detected on or within surfaces, substances or products treated according to the methods of this invention.

For the purposes of this invention, the language “limiting the growth and/or the presence of a virus, bacterium and fungus” as used herein refers to methods that employ the use of the compounds described in this invention to inhibit, kill, prevent the replication of or reduce the number of viruses, bacteria or fungi present on a surface, substance or product exposed to the compounds described in this invention. For experimental purposes, growth of bacteria or fungi is limited when a particular bacterium or fungus is growth inhibited or killed when exposed to the compounds of this invention. For example, growth of bacteria or fungi is limited by the compounds of this invention when disks moistened with a solution containing the compounds of this invention create visible zones of growth inhibition on a surface containing the bacteria or fungi.

Furthermore, some of the embodiments of the present invention are directed to inhibiting a rho transcription termination factor, wherein the method comprises administering a chelating agent and a compound comprising at least one thiol group. The chelating agent may be divalent metal or metal oxide. Rho is an essential protein found in most Gram-negative bacteria, including, but not limited to Escherichia coli, shigella, salmonella, citrobacter, enterobacter cloacae, neisseria gonorrhoeae and helicobacter pylor. Furthermore, recent studies have shown that rho also plays a pivotal role in the gram-positive bacterium, micrococcus luteus. Additionally, BLAST analysis has indicated that rho has a likely presence in Bacillus anthracis. Thus, the potential of a macromolecular (protein) selective targeting agent such as the present compounds provides an important opportunity for new drug development.

A. Transcription Termination Factor Rho

The transcription termination factor rho is an essential protein necessary for Gram-negative bacterial growth. Yager et al., (1987) In The Molecular and Cell Biology of E. coli and S. typhimurium, (Neidhardt, F., Ed.) Am. Soc. Microbiol., pp. 1241-1275, Washington, D.C. Despite the rho protein's central importance in the cell biology of these organisms, the only reported selective inhibitor for this enzyme is the natural product, bicyclomycin. Zwiefka et al., (1993) Transcription termination factor rho: The site of bicyclomycin inhibition in Escherichia coli, Biochemistry 32, 3564-3570. Some of the embodiments of the present invention may illustrate that select metal chelates inhibit rho function by a unique pathway and that the inhibitory activities of these chelates can exceed that of bicyclomycin.

Transcription termination begins by recognition and binding rho to specific RNA sequences known as rut (rho utilization) sites. The initial site of rho-binding corresponds to the primary RNA-binding domain. Translocation is believed to occur via a tethered-tracking mechanism in which rho remains bound to RNA at the primary binding site and makes advancing contacts with RNA at the secondary RNA binding site. Secondary site binding may occur by the hexamer's opening up to permit RNA passage into the solvent-exposed central hole or its forming around the RNA transcript. ATP hydrolysis fuels the 5′ to 3′ movement of rho along the RNA toward the stalled polymerase. The transcript than may dissociate from the polymerase when rho encounters the polymerase, possibly by a rho helicase activity. It is presumed that rho is freed from the RNA by subunit dissociation (disruption).

In 1964, Cleland showed that dithiothreitol (DTT, 2a) and its isomer dithioerythreitol (DTE, 2b) quantitatively converted disulfides to their corresponding thiols. Cleland, (1964) Dithiothreitol, a new protective group for SH groups, Biochemistry 3, 480-482.

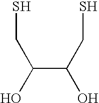

dithiothreitol (DTT, 2a)

These 1,4-dithio-2,3-dihydroxybutanes (DTX) have become the reagents of choice to protect sulfhydryl groups in enzymes.

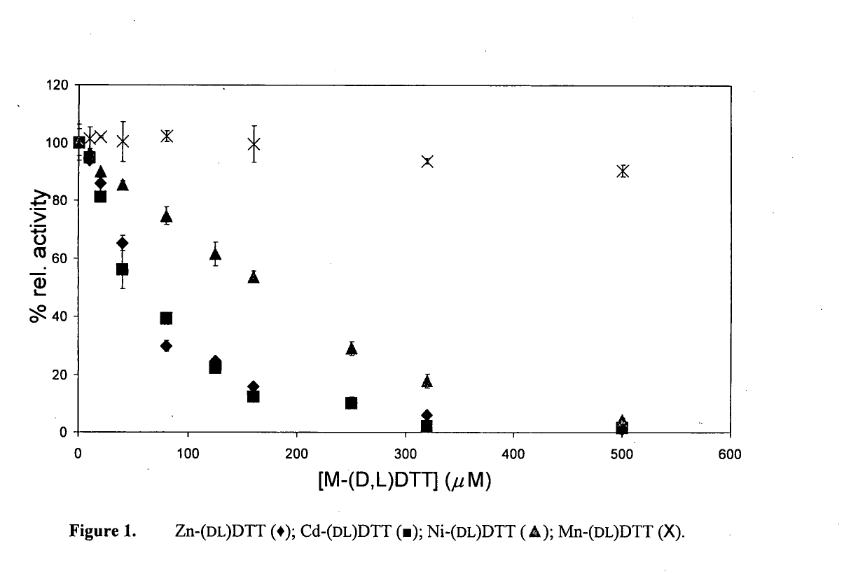

Equimolar (DL)DTT solutions containing either ZnCl₂, CdCl₂, or NiCl₂ inhibited rho poly(C)-dependent ATP hydrolysis in the presence of MgCl₂. Significantly, these metals stimulated rho ATPase activity in the absence of MgCl₂ and (DL)DTT. Weber et al., (2003) Metal dependency for transcription factor rho activation, Biochemistry 42, 1652-1659. By comparison, (DL)DTT (0.1-0.3 mM) by itself or in combination with either MgCl₂ or MgCl₂ (FIG. 1) did not inactivate the enzyme. In some of the embodiments of the present invention, rho inhibition was observed when the metal and chelator were both present.

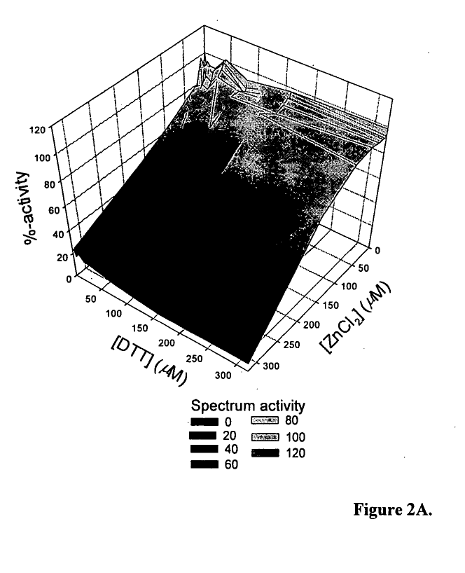

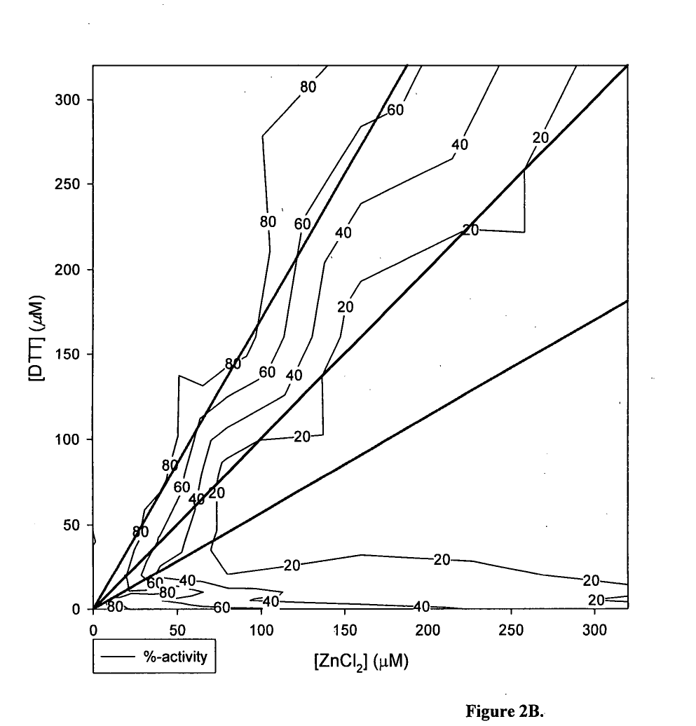

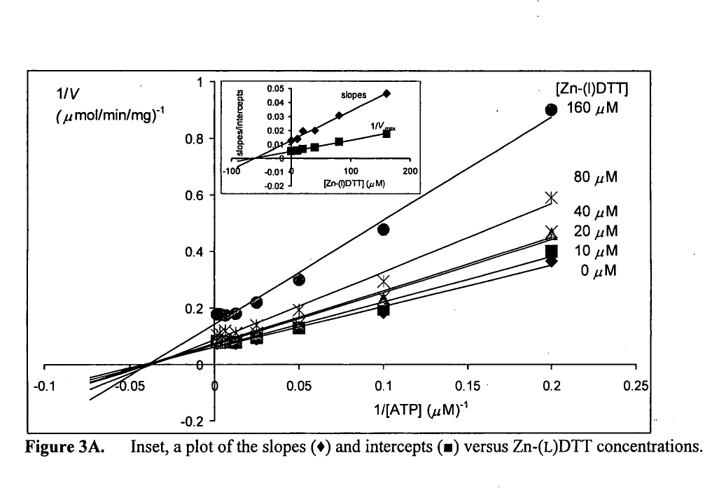

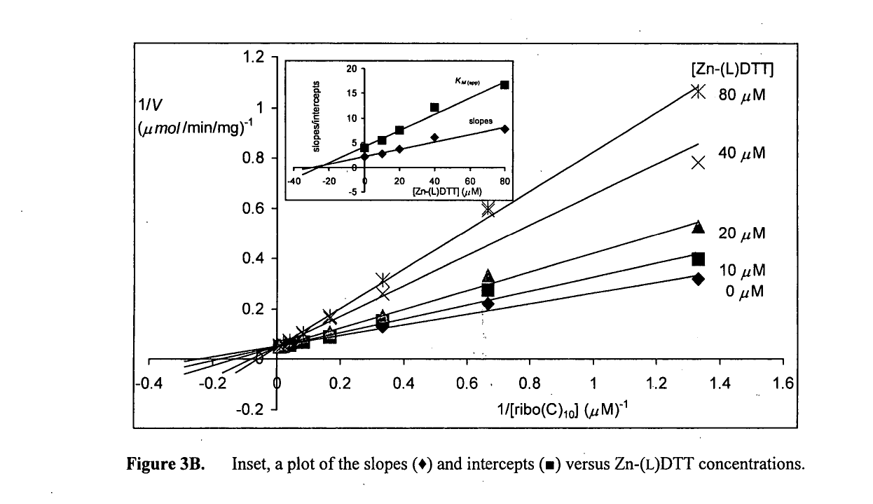

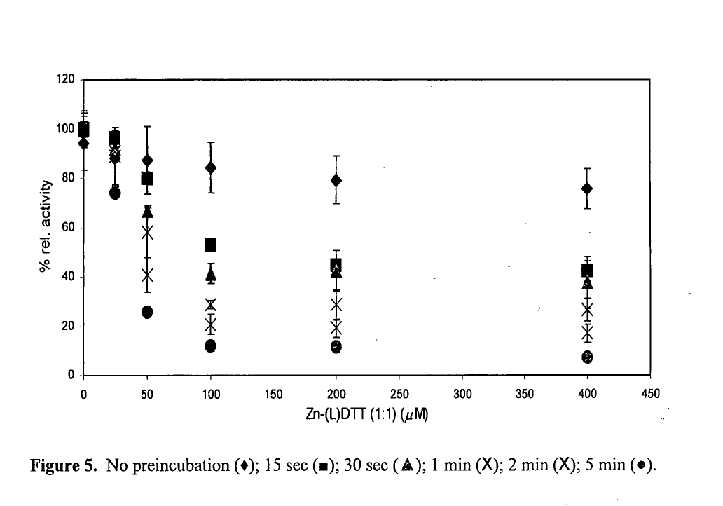

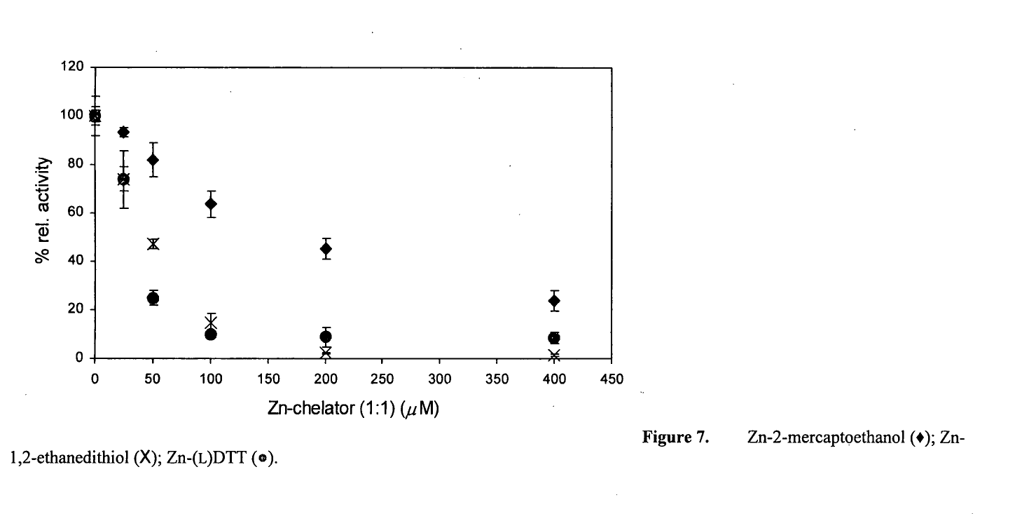

Some of the embodiments of the present invention include the examination of a ZnCl₂-(L)DTT inhibition and a focus on the structure of the inhibitory complex. The rho poly(C)-dependent inhibitory activities of equimolar solutions of ZnCl₂ and either (L)DTT (I₅₀=45 μM) or ethanedithiol (I₅₀=50 μM) were comparable and were significantly higher than an equimolar solution of ZnCl₂ and 2-mercaptoethanol (I₅₀=175 μM), which suggests that metal-to-DTT binding preferentially occurred through sulfur rather than oxygen (FIG. 7). There was little differences in the extent of rho inhibition for the Zn-chelates of DTT and DTE, thus demonstrating that the stereochemical disposition of the hydroxy groups did not affect rho inhibition. The stoichiometry for maximal rho poly(C)-dependent ATPase inhibition corresponded with Zn₂-(L)DTT (FIG. 2). This stoichiometry differed from the previously identified Zn-(DL)DTT, Zn-((DL)(DTT))₂, and Zn₃-((DL)(DTT))₄ complexes. Krezel et al., (2001) Coordination of heavy metals by dithiothreitol, a commonly used thiol group protectant, J. Inorg. Biochem. 84, 77-78. Chelate inhibition was observed and required a 2-min preincubation period at 32° C. (FIG. 5) thereby illustrating that the structure of the Zn-(DL)DTT rho inhibitory species may be affected by protein binding. Although slow binding inhibitory processes may proceed through changes in protein conformation, the preincubation of 2 min was sufficient to obtain kinetic data that followed a linear response when plotting the slope and intercepts from the double reciprocal plots (FIG. 3A). Moreover, after two min preincubation further slowing of the velocity curves was not observed, suggesting that steady state rates were being achieved within two minutes.

The mechanism of Zn-(L)DTT inhibition of rho was also investigated. The Zn-(L)DTT complex (1:1) showed noncompetitive kinetics with respect to ATP in the poly(C)-dependent ATPase assay (FIG. 3A) and fully competitive kinetics with respect to ribo(C)₁₀ in the poly(dC)-ribo(C)₁₀ ATPase assay (FIG. 3B). These results may indicate that the site of Zn-(L)DTT inhibition is close to or at the rho secondary RNA-binding site. Xu et al., (2002) Mutations in the rho transcription termination factor that affect RNA tracking, J. Biol. Chem. 277, 30023-30030; see also, Wei et al., (2001). Mutational changes of conserved residues in the Q-loop region of transcription factor rho greatly reduce secondary site RNA-binding, J. Mol. Biol. 314, 1007-1015. The kinetic patterns for Zn-(L)DTT differentiated this inhibitor from bicyclomycin where reversible, noncompetitive inhibition was observed with respect to ATP in the poly(C)-dependent ATPase assay, but mixed inhibition was observed with respect to ribo(C)₁₀ in the poly(dC)-ribo(C)₁₀ ATPase assay. Magyar et al., (1996); see also, Park et al., (1995) Bicyclomycin and dihydrobicyclomycin inhibition kinetics of Escherichia coli rho-dependent transcription termination factor ATPase activity, Arch. Biochem. Biophys. 323, 447-454. Thus, while the two inhibitors disrupt ATP hydrolysis, they function by different pathways.

Some of the embodiments of the present invention documents the potent inhibitory activity of Cd²⁺, Ni²⁺, and Zn²⁺-DTT chelates and provide the basis for a new class of site-specific rho inhibitors. Different coordination properties of metals and the manifold structural properties of sulfur, oxygen and nitrogen ligands will lead to the discovery of other rho inhibitors.

In order that this invention may be better understood, the following examples are set forth. These examples are for purposes of illustration only, and are not to be construed as limiting the scope of the invention in any manner.

Materials and Methods

Materials. Bicyclomycin was provided as a gift from Fujisawa Pharmaceutical Co., Ltd. (Osaka, Japan) and was purified by three successive silica gel chromatographies using 20% methanol-chloroform as the eluant. [γ-³²P] ATP (6000 Ci/mmol) was purchased from Perkin Elmer (Boston, Mass.), Bio-Spin 6 columns were from Bio-Rad (Hercules, Calif.), and PEI-TLC plates used for ATPase assays were obtained from J.T. Baker, Inc. (Phillipsburg, N.J.). Poly(C) was from Sigma (St. Louis, Mo.) and was dissolved in 100 μL of TE buffer and dialyzed against aqueous 1.0 M potassium phosphate, pH 7.0 (8 h, 2×, 4° C.) using Slide-A-Lyzer cassettes from Pierce (Rockford, Ill.). All other chemicals were reagent grade.

Bacterial strains and plasmids. Wild-type rho from E. coli was purified as described by Mott from strain AR120 containing plasmid p39-ASE. Nehrke et al., (1992); see also, Mott et al., (1985) Maximizing gene expression from plasmid vectors containing the λ P_{L} promoter: Strategies for overproducing transcription termination factor ρ, Proc. Natl. Acad. Sci. U.S.A. 82, 88-92. Overproduced rho factor from p39AS has lysine replacing glutamic acid at residue 155 in the linker region between its RNA and ATP binding domains, Nucleic Acids Res. 20, 6107. Rho purity was determined by SDS-PAGE, and protein concentration was measured according to the Lowry assay. Lowry et al., (1951) Protein measurement with the folin phenol reagent, J. Biol. Chem. 193, 265-275.

Rho poly(C)-dependent ATPase assay. See, generally, Sharp et al., (1983) A kinetic mechanism for the poly(C)-dependent ATPase of the Escherichia coli transcription termination protein, rho, J. Biol. Chem. 258, 3482-3486. The ribonucleotide-stimulated ATPase activity of rho at 32° C. was assayed by the amount of ³²P-labeled inorganic phosphate hydrolyzed from ATP after separation on PEI-TLC plates (prerun with water and dried) using 0.75 M potassium phosphate (pH 3.5) as the mobile phase. Reactions were initiated by adding ATP (varying concentrations) and 0.5 μCi [γ-³²P] ATP to the solution containing 40 mM Tris-HCl (pH 7.9), 50 mM KCl, 100 nM or 250 nM poly(C), 100 or 250 nM rho (monomer) and MgCl₂ (1 mM or 10 mM). The TLC plates were exposed to PhosphorImager plates (Fuji and Molecular Dynamics) (3 h), scanned on a Storm 860 PC PhosphorImager and analyzed using Molecular Dynamic's ImageQuant 5.0. The initial rates of the reactions were determined by plotting the amount of ATP hydrolyzed against time. Each reaction was performed in duplicate and the results averaged.

Inhibition of rho poly(C)-dependentATPase activity by metal (D,L)DTT chelates. ATPase assays were carried out as described in the preceding section using a six-channel, multiwell procedure. Weber et al., (2003) Metal dependency for transcription factor rho activation, Biochemistry 42, 1652-1659. The reactions (100 μL) were run with 250 μM ATP in the presence of the metal-(DL)DTT (1:1) solution (0-500 μM) and 1 MM MgCl₂. Samples were preincubated at 32° C. for 2 min and then ATP was added. Aliquots (5 1.4-μL) were spotted onto PEI-TLC plates after 15 s and then at selected times (10-300 s) thereafter. Plates were analyzed as described previously. Each reaction was performed in duplicate and the results averaged.

Inhibition of rho poly(C)-dependent ATPase activity by Zn-(L)DTT. The stoichiometry of the inhibitor. The poly(C)-dependent ATPase assays were carried out as described in the preceding section using an eight-channel, multiwell procedure Weber et al., (2003). The duplicated reactions (100 μL) were run with 250 μM ATP and 10 mM MgCl₂ in the presence of 0, 2.5, 5, 10, 20, 40, 80, 160 and 320 μM ZnCl₂. Each of the preceding experiments was then repeated in the presence of 0, 2.5, 5, 10, 20, 40, 80, 160 and 320 μM (L)-DTT resulting in a 9×9 reaction matrix. Samples were preincubated at 32° C. for 2 min prior to ATP addition, and 5 1.4-μL aliquots were spotted onto PEI-TLC plates every 15 s. The plates were treated as described previously. The data were analyzed using Sigmaplot 2001 using the XYZ triplet line plot function and plotted as a color-coded, 3D-mesh and contour plot (FIG. 2A). The fractional inhibition (0.1, 0.2, . . . , 0.9) in the contour plots were determined from the inhibition curves. Concentration combinations of Zn²⁺ and (L)DTT that gave equal fractional inhibition were plotted (FIG. 2B).

Kinetics of rho poly(C)-dependent ATPase activity and inhibition by Zn-(L)DTT (1:1). ATPase assays were carried out as described in the preceding section using a six-channel, multiwell procedure. Weber et al., (2003). The reactions (100 μL) were run with 2.5, 5, 10, 20, 40, 80, 160, 320, and 640 μM ATP in the presence of 10 mM MgCl₂. Each series was repeated in the presence of 10, 20, 40, 80 or 160 μM Zn-(L)DTT (1:1). Samples were preincubated at 32° C. for 2 min prior to ATP addition and 5 1.4-μL aliquots were spotted onto PEI-TLC plates after 15 s and then at selected times (10-300 s), depending upon the ATP concentrations. Plates were analyzed as described previously. Each reaction was performed in duplicate and the results averaged.

Rho poly(dC)-ribo(C)₁₀-dependent ATPase assay. See, generally, Richardson et al., (1982) Activation of rho protein ATPase requires simultaneous interaction at two kinds of nucleic acid-binding sites, J. Biol. Chem. 257, 5760-5766. The ribonucleotide-stimulated ATPase activity of rho at 32° C. was assayed by the amount of ³²P-labeled inorganic phosphate hydrolyzed from ATP after separation on PEI-TLC plates (prerun with water and dried) using 0.75 M potassium phosphate (pH 3.5) as the mobile phase. Reactions were initiated by adding 200 μM ATP and 0.5 μCi [γ-³²P] ATP to the solution containing 40 mM Tris-HCl (pH 7.9), 50 mM KCl, 200 nM poly(dC), 100 nM rho (monomer), ribo(C)₁₀ (various concentrations), and 10 mM MgCl₂, and 5 1.4-μL aliquots were spotted onto PEI-TLC plates every 15 s. Plates were analyzed as described previously. Each reaction was performed in duplicate and the results averaged.

Kinetics of rho poly(dC)-ribo(C)₁₀-stimulated ATPase activity and inhibition by Zn-(L)DTT (1:1). ATPase assays were carried out as described in the preceding section using a six-channel, multiwell procedure. Weber et al., (2003). The reactions (30 μL) were run with 0.75, 1.5, 3, 6, 12, 24, 48, and 96 μM ribo(C)₁₀. Each series was repeated in the presence of 10, 20, 40, and 80 μM of Zn-(L)DTT. Samples were preincubated at 32° C. for 2 min prior to ATP addition, and 5 1.4-μL aliquots were spotted onto PEI-TLC plates after 15 s and then at selected times (10-300 s) thereafter. Plates were analyzed as described previously. Each reaction was performed in duplicate and the results averaged.

Promiscuity tests for Zn-(L)DTT (1:1) and bicyclomycin. The rho poly(C)-dependent ATPase activity measurements were conducted using a solution (100 μL) containing rho (100 nM (monomer)), poly(C) (100 nM), ATP (200 μM), MgCl₂ (10 mM) and either Zn-(L)DTT (1:1) (0-800 μM) or bicyclomycin (0-400 μM) at 32° C. for a 2-min preincubation. The average velocities of two determinations are plotted. Each of the preceding experiments was then repeated as follows: the preincubation times were varied, the rho and the poly(C) concentration were increased to 1 μM, and the reaction contained 0.1 mg/mL BSA. The reactions were analyzed as described above.

Inhibition of rho poly(C)-dependent ATPase activity by Zn-chelates. The poly(C)-dependent ATPase assays were carried out as described in the preceding section using a six-channel, multiwell procedure. Weber et al., (2003). The reactions (100 μL) were run with 200 μM ATP in the presence of 0, 25, 50, 100, 200, and 400 μM of the Zn-chelate (1:1) (Zn-2-mercaptoethanol, Zn-1,2-ethanedithiol). Samples were preincubated at 32° C. for 2 min prior to ATP addition, and 5 1.4-μL aliquots were spotted onto PEI-TLC plates every 15 s. Plates were analyzed as described previously. Each reaction was performed in duplicate and the results averaged.

The present invention will now be described with reference to the following examples. It should be appreciated that these examples are for the purposes of illustrating aspects of the present invention, and do not limit the scope of the invention as defined by the claims.

### EXAMPLES

CdCl₂, MnCl₂, NiCl₂, and ZnCl₂ in (DL)DTT-free buffer solutions all activated rho poly(C)-dependent ATP hydrolysis in the absence of MgCl₂. Surprisingly, after adding an equimolar amount of (DL)DTT to solutions containing either CdCl₂, NiCl₂, or ZnCl₂ in the presence of MgCl₂ (1 mM) a pronounced loss of rho activity was observed (I₅₀ (μM): CdCl₂, 60; NiCl₂, 160; and ZnCl₂, 55; FIG. 1). An earlier study reported that CdCl₂ and ZnCl₂ rho solutions maintained in buffer solutions containing (DL)DTT inhibited rho and that the inhibitory species was the divalent metal; see, Lowery et al., (1977) Characterization of the nucleoside triphosphate phosphohydrolase (ATPase) activity of RNA synthesis termination factor ρ. I. Enzymatic properties and effects of inhibitors, J. Biol. Chem. 252, 1375-1380. But (DL)DTT (0.1-0.3 mM) alone or in combination with either MnCl₂ (FIG. 1) or MgCl₂ did not inactivate the enzyme. This illustrates that the DTT-mediated inactivation process may be metal specific.

The studies were focused on ZnCl₂ with DTX and defined the parameters that governed metal chelate inhibition of rho poly(C)-dependent ATPase activity. Data was obtained after preincubating (2 min, 32° C.) the inhibitor, rho, poly(C) in the buffer solution prior to adding ATP to initiate the assay. First the effect of DTX stereochemistry was examined on rho poly(C)-dependent ATPase inhibition. The three stereoisomers for DTX are (D)DTT, (L)DTT, and the meso-form, dithioerythritol (DTE). (L)DTT, (DL)DTT, and DTE are commercially available. Minor differences in the I₅₀ values were observed for the 1:1 ZnCl₂-DTX solutions (0-800 μM) as the chelator (was varied I₅₀ (μM): Zn-(DL)DTT, 60; Zn-(L)DTT, 50; and Zn-DTE, 60) in the presence of 10 mM MgCl₂. This finding may indicate that the inhibitor-protein interaction was independent of the DTX configuration.

Next, the ZnCl₂ to DTT stoichiometry was determined for maximal inhibition. DTT has been reported to give metal complexes with different stoichiometries depending on the metal-to-DTT ratio and their solution concentrations. Krezel et al., (2001) Coordination of heavy metals by dithiothreitol, a commonly used thiol group protectant, J. Inorg. Biochem. 84, 77-78. A 9×9 matrix experiment was conducted in which both the ZnCl₂ and the (L)DTT concentrations ranged from 0-320 μM (FIG. 2A). The data showed that maximal inhibition (decreased rho activity) occurred when the ZnCl₂-to-(L)DTT ratio was 2:1 (FIG. 2B, red line) and that inhibitory activity diminished as the ZnCl₂-to-(L)DTT ratio approached 1:2 (FIG. 2B, blue and green lines). The I₅₀ value for the 2:1 Zn-(L)DTT solution was 20 μM, which was three times more potent than bicyclomycin (I₅₀=60 μM). Zwiefka et al., (1993); see also, Park et al., (1995) Bicyclomycin and dihydrobicyclomycin inhibition kinetics of Escherichia coli rho-dependent transcription termination factor ATPase activity, Arch. Biochem. Biophys. 323, 447-454.

The mechanism of Zn-(L)DTT (1:1) inhibition of rho ATP hydrolysis with respect to both ATP and ribo(C)₁₀ was also examined. FIG. 3A shows the double, reciprocal plot of 1/V versus 1/[ATP] at various Zn-(L)DTT (1:1) concentrations in the poly(C)-dependent ATP hydrolysis assay (10 mM MgCl₂). The graph indicates that the Zn-(L)DTT (1:1) solution inhibited the conversion of ATP to ADP and Pᵢ by a noncompetitive pathway, with respect to ATP. When the intercepts and slopes of the double, reciprocal graphs were plotted against the Zn-(L)DTT (1:1) concentrations, straight lines (FIG. 3A, inset) were obtained suggesting that the inhibitor bound at a unique, non-interacting site with a Kᵢ of 60 μM. A similar kinetic finding has been reported for bicyclomycin (Kᵢ=20 μM). Park et al., (1995) Bicyclomycin and dihydrobicyclomycin inhibition kinetics of Escherichia coli rho-dependent transcription termination factor ATPase activity, Arch. Biochem. Biophys. 323, 447-454. FIG. 3B depicts the inhibition kinetics for Zn-(L)DTT (1:1) solutions in the poly(dC)-ribo(C)₁₀-stimulated ATPase assay (10 mM MgCl₂). The Zn-(L)DTT (1:1) solution was found to be a competitive inhibitor with respect to ribo(C)₁₀. This finding differed from bicyclomycin, which showed a mixed inhibition pattern. Magyar et al., (1996). The K_{M} for ribo(C)₁₀ was measured as 4 μM in the absence of inhibitor and increased to 20 μM at 80 μM Zn-(L)DTT. For the Zn-(L)DTT (1:1) solution, a Kᵢ of 28 μM was observed (FIG. 3B, inset). Thus, bicyclomycin and Zn-(L)DTT (1:1) both disrupted rho-mediated ATP hydrolysis, but they functioned by different pathways.

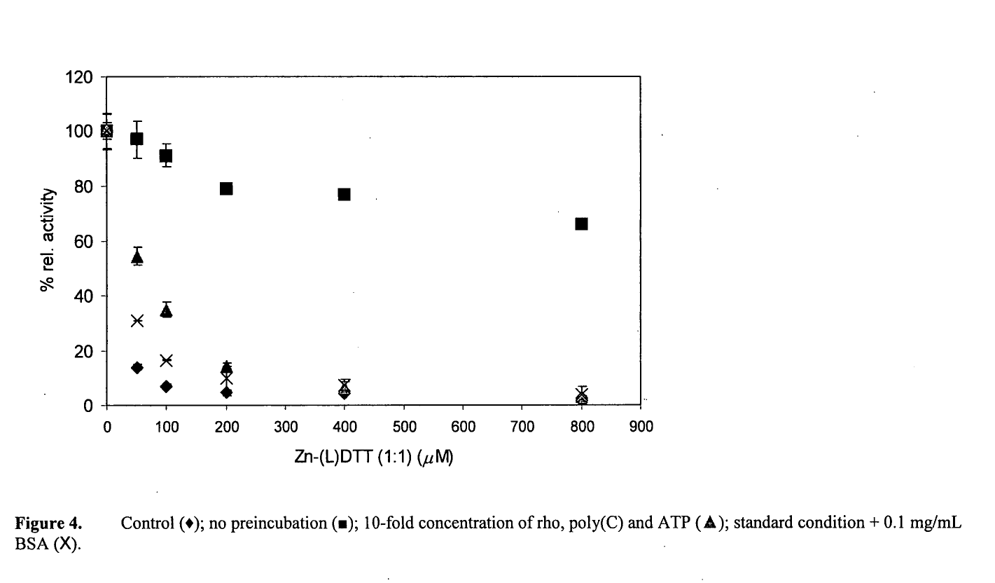

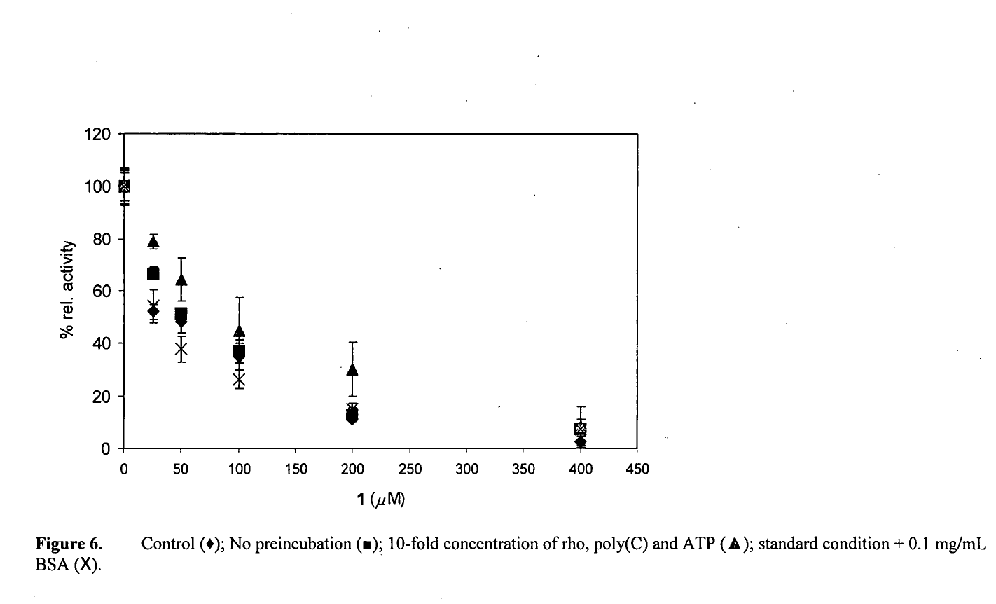

With the use of high-throughput and virtual screening techniques the number of new lead compounds for drug development has dramatically increased. The validity of some of these new leads has been questioned, and attention is now focused on determining the inhibition pathway for these compounds and whether the inhibitors show enzyme specificity. McGovern et al., (2001) A common mechanism underlying promiscuous inhibitors from virtual and high-throughput screening, J. Med. Chem. 45, 1712-1722. Compounds that show nonspecific interaction have been defined as “promiscuous inhibitors.” The inhibitor specificity of Zn-(L)DTT complexes was tested. The I₅₀ (30 μM) value for Zn-(L)DTT (1:1) solutions was not affected appreciably either by adding BSA (0.1 mg/mL) (I₅₀=40 μM) or by a 10-fold increase in rho concentration (I₅₀=60 μM) (FIG. 4). Next, the time dependency for chelate inhibition was examined because a well-behaved, reversible inhibitor is typically not affected by incubation time. McGovern et al., (2001). Maximal inhibition was observed for Zn-(L)DTT after preincubation of the reaction mixture, other than ATP, for 2 min. Without preincubation, no loss of rho activity was observed (FIGS. 4 and 5). Bicyclomycin was tested in a similar manner and its I₅₀ value (50 μM) was not significantly affected when BSA (0.1 mg/mL) was included, when rho concentration increased (10-fold), or when inhibitor preincubation time was reduced to 0 (FIG. 6). The effect of Zn-(L)DTT solutions on E. coli RecA poly (dT)-dependent ATP hydrolysis was determined. The addition of an equimolar amount of (L)DTT to the ZnCl₂ solution did not cause further reductions in the rate of ATP hydrolysis, indicating that Zn-(L)DTT complexes did not inhibit RecA poly(dT)-dependent ATP hydrolysis. Similarly, no loss in RecA ATPase activity was observed when bicyclomycin (0-400 μM) was included in the reaction solution. These collective findings indicate that both Zn-(L)DTT and bicyclomycin were selective, non-promiscuous inhibitors and that Zn-(L)DTT (1:1) binding to rho was either slow or the chelating species underwent structural reorganization prior to binding.

Several attempts were made to determine if Zn-(L)DTT (1:1) inhibited bacterial growth. Using W3350 E. coli and either a filter disc or a liquid culture assay the apparent precipitation of the inhibitor with no loss of bacterial growth was observed when Zn-(L)DTT (1:1) was added to the medium. Ericsson et al., (1971) Antibiotic sensitivity testing. Report of an international collaborative study, Acta Pathol. Microbiol. Scand. Section A, Suppl. 217, 1-90. For a similar protocol, see: Kim et al., (2003) N(2)-Substituted D,L-cycloserine derivatives: Synthesis and evaluation as alanine racemase inhibitors, J. Antibiot. 56, 160-168.

Next, it was determined whether ZnCl₂ solutions containing either 1,2-ethanedithiol or 2-mercaptoethanol inhibited rho poly(C)-dependent ATPase activity and compared the results with (L)DTT. The I₅₀ values for ZnCl₂ solutions containing equimolar amounts of either (L)DTT, 1,2-ethanedithiol, or 2-mercaptoethanol in the presence of 10 mM MgCl₂ were 45 μM, 50 μM, and 175 μM, respectively (FIG. 7). Thus, it may be tentatively concluded that Zn-dithiol chelates are more effective than Zn-mercaptoalkanol adducts in inhibiting rho.

### Example 2

### Metal Dependency

The potential metal-dependent profiles can be grouped into the four patterns previously predicted by London and Steck. London et al. (1969) Kinetics of enzyme reactions with interaction between a substrate and a (metal) modifier, Biochemistry 8, 1767-1779. First are hyperbolic metal activation curves. These curves show Michaelis-Menten kinetics and contain one Vₘₐₓ and one K_{M} value for the substrate M·ATP²⁻, where M²⁺ can be any divalent cation. Second are metal activation curves that increase with increasing metal concentration and then decrease as the metal concentrations further increase. These velocity curves exhibit a peak velocity, Vₚₑₐₖ. Third are activation curves that show a sigmoidal shape for the ATP hydrolysis rate versus total M²⁺ concentration. The sigmoidal shape provides information about cooperative processes for ATP hydrolysis where metal binding at one site either enhances or diminishes ATP hydrolysis at another site. However, when interpreting sigmoidal curves for metal activation it may be important to take special care because metals can randomly bind to protein or other biomolecules leading to similarly shaped activation curves. Fourth are experimental plots that show no activation with increasing metal concentrations. This case refers to metals that are incapable of catalyzing the hydrolysis of ATP or that may actually inhibit the reaction. Finally, the second and third kinetic patterns can be combined where a peak velocity is observed, but only if the initial activation curve is sigmoidal.

A. Divalent Metal Activation of Rho in the Absence of MgCl₂. The roles of the 11 divalent metals (Be²⁺, Ca²⁺, Cd²⁺, Co²⁺, Cu²⁺, Hg²⁺, Mn²⁺, Ni²⁺, Sr²⁺, VO²⁺, Zn²⁺) were investigated in rho-dependent processes and then compared with Mg²⁺, the metal cofactor. For each metal, wild-type rho was pretreated with 20 mM EDTA to deplete the protein of residual metals and removed the excess EDTA and the metal•EDTA complex by a spin column. It was determined that rho retained 15% or less of residual poly(C)-dependent ATPase activity, and upon addition of 10 mM MgCl₂, ˜90% activity of the untreated sample was recovered. The rates of ATP hydrolysis were measured at 12 divalent metal ion concentrations (1.9, 3.8, 7.8, 15.6, 31.3, 62.5, 125, 250, 500, 1000, 2000, 4000 μM), and the residual (≦15%) ATPase activity from the spin column treated rho was subtracted from the observed activity.

The results indicated that the divalent metals can be grouped into four classes based on their metal activation profiles. First, Mg²⁺, Ca²⁺, Co²⁺, Cu²⁺, Hg²⁺, and VO²⁺ exhibited Michaelis-Menten kinetics. Cd²⁺, Mn²⁺, and Zn²⁺ fall in the second group showing peak velocities at different metal concentrations. Within this category, Cd²⁺ and Zn²⁺ showed curves with sigmoidal behavior. The third group contained only Ni²⁺, which showed a sigmoidal activation pattern rising to a constant Vₘₐₓ at high metal concentrations. Be²⁺ and Sr²⁺ lie in the fourth group and did not catalyze ATP hydrolysis, and thus no activation profiles were observed.

For the first group, maximal ATP hydrolysis rates were observed at metal concentrations of 250 μM and higher, and the order of activation was Mg²⁺>Co²⁺>Ca²⁺>Cu²⁺˜Hg²⁺˜VO²⁺. Within this group, the best activator, Mg²⁺, hydrolyzed ATP nearly 10 times faster than the three poorest activators, Cu²⁺, Hg²⁺, and VO²⁺.

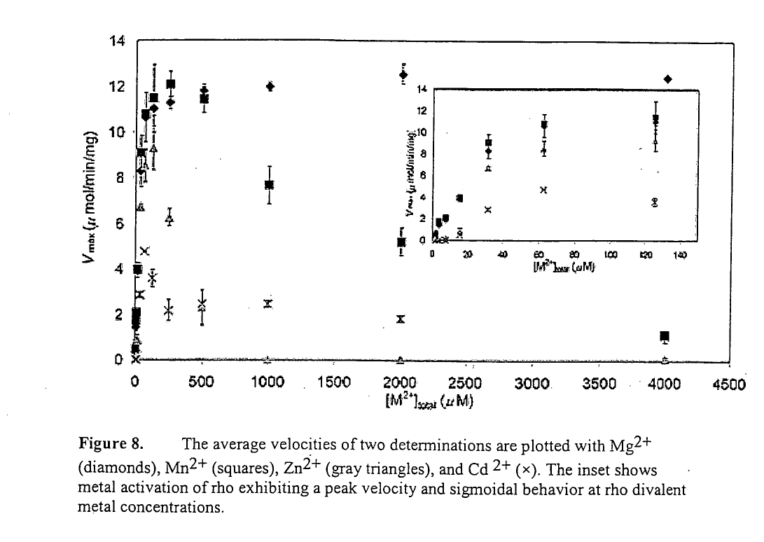

Three metals, Cd²⁺, Mn²⁺, and Zn²⁺, showed peak velocities when concentrations were progressively increased from 1.9 to 4000 μM (FIG. 8). The Mg²⁺ activation curve is included in the figure for comparison. The peak velocity for Mn²⁺ (11.9 μmol/min/mg) was observed at 250 μM, and the Vₚₑₐₖ was found to be similar to the Mg²⁺ Vₘₐₓ, making both metals equally effective in hydrolyzing ATP at low metal concentrations. Above 250 μM [Mn²⁺]ₜₒₜₐₗ the velocity rapidly diminished, reaching 10% of maximum activity at 4000 μM. The peak velocity of Zn²⁺ was 9.4 μmol/min/mg at a [Zn²⁺]ₜₒₜₐₗ of 125 μM and then decreased to zero at 1000 μM. A hyperbolic loss in rho activity was observed beyond 125 μM [Zn²⁺]ₜₒₜₐₗ, and at 190 μM [Zn²⁺]ₜₒₜₐₗ only 50% of the activity remained. The hyperbolic drop of the ZnCl₂ curve beyond the peak velocity suggested that inhibition occurred by the divalent metal binding to a specific site. Finally, for Cd²⁺ the peak velocity of 4.8 μmol/min/mg was reached at 65 μM [Cd²⁺]ₜₒₜₐₗ and slowly tapered off over the higher concentration range. Cd²⁺ and Zn²⁺ exhibited a sigmoidal shape in the hydrolysis rate at low metal concentrations.

In Table 1, the dissociation constant (K_{D}) was summarized for each M•ATP complex at 25° C. along with the observed maximal velocities for each metal, the K_{M(app)}, and the Hill constants for the velocity curves, and the Vₘₐₓ/K_{M} and Vₚₑₐₖ/K_{M} values were calculated. These numbers are a measure of the efficiency of each metal in rho-mediated ATP hydrolysis. The larger the V/K_{M} value, the more efficient the enzyme in hydrolyzing ATP.

TABLE 1
Summary of the Divalent Metals for Rho Activation

| | | Vₘₐₓ₍ₐₚₚ₎^{b} | Vₚₑₐₖ₍ₐₚₚ₎^{b} | | | |
|---|---|---|---|---|---|---|
| | K_{D} | (mol | (mol | | V/K_{M(app)}^{d} | |
| | (MATP)^{a} | min⁻¹ | min⁻¹ | K_{M(app)}^{c} | (×10³ | n |
| M²⁺ | (M) | mg⁻¹) | mg⁻¹) | (M) | min⁻¹) | (Hill)^{e} |
| Mg²⁺ | 60.2 | 12.3 | −/− | 21.2 | 579 | 1 |
| Be²⁺ | −/− | 0.21 | −/− | n.d.^{f} | n.d. | n.d. |
| Ca²⁺ | 107 | 3.13 | −/− | 7.3 | 428 | 1 |
| Cd²⁺ | 43.6 | −/− | 4.74 | 41.8 | 114 | 4.6 |
| Co²⁺ | 21.9 | 7.65 | −/− | 21.8 | 351 | 1 |
| Cu²⁺ | 0.7 | 1.54 | −/− | 6.8 | 227 | 1 |
| Hg²⁺ | −/− | 0.78 | −/− | 4.7 | 167 | 1 |
| Mn²⁺ | 16.6 | −/− | 11.9 | 23.1 | 513 | 1 |
| Ni²⁺ | 9.5 | 4.79 | −/− | 28.2 | 170 | 2.9 |
| Sr²⁺ | 288 | 0.17 | −/− | n.d. | n.d. | 1 |
| VO²⁺ | 0.2 | 0.78 | −/− | n.d. | n.d. | 1 |
| Zn²⁺ | 14.1 | −/− | 9.98 | 33.6 | 297 | 5.5 |
| none | −/− | 0.70 | −/− | n.d. | n.d. | n.d. |

^{a}K_{D}(MATP) = [M²⁺] · [ATP]/[MATP] at 25° C. for Mg²⁺, Mn²⁺, Zn²⁺, Ni²⁺, Cd²⁺, Ca²⁺, Co²⁺, Cu²⁺, VO²⁺,Sr²⁺.
^{b}Vₘₐₓ or Vₚₑₐₖ were determined from the activation curves.
^{c}K_{M} determined by estimating total M²⁺concentration at 0.5 · Vₘₐₓ or 0.5 · Vₚₑₐₖ.
^{d}V/K_{M} is a measure for substrate specificity and efficiency.
^{e}The Hill constant was determined through nonlinear regressional analysis (SigmaPlot 2001).
^{f}n.d. = not determined because the values were too low to be accurately calculated.

TABLE 2
Effect of Metals on Rho Activity^{a}

| | Mg²⁺-free | | |
|---|---|---|---|
| | % activity | % efficiency | 10 mM |
| metal | (Vₘₐₓ or Vₚₑₐₖ) | (Vₘₐₓ/K_{M} or Vₚₑₐₖ/K_{M}) | MgCl₂ |
| Mg²⁺ | 100 (e) | 100 (e) | n.d. |
| Be²⁺ | 2 (i) | n.d. | i |
| Ca²⁺ | 25 (w) | 74 (g) | n |
| Cd²⁺ | 39 (m) | 20 (w) | n |
| Co²⁺ | 62 (g) | 61 (g) | n |
| Cu²⁺ | 13 (p) | 39 (m) | i |
| Hg²⁺ | 6 (p) | 29 (w) | i |
| Mn²⁺ | 97 (e) | 89 (e) | n.d. |
| Ni²⁺ | 39 (m) | 29 (w) | n |
| Sr²⁺ | 1 (n) | n.d. | n |
| VO²⁺ | 6 (p) | n.d. | n |
| Zn²⁺ | 81 (g) | 51 (m) | i |

^{a}e = excellent activator; g = good activator; m = moderate activator; w = weak activator; p = poor activator; n = nonactivating metal; i = inhibitor; n.d. = not determined.

No correlation was observed in either the maximal velocity for ATP hydrolysis (Vₘₐₓ or Vₚₑₐₖ) or the efficiency of ATP hydrolysis (V/K_{M}) values with the K_{D} for the M•ATP²⁻ complex. Of the three metals that bound ATP the most tightly (VO²⁺, Cu²⁺, Ni²⁺), only Ni²⁺ gave significant ATP hydrolysis. Correspondingly, metals that bound ATP poorly displayed excellent (Mg²⁺), moderate (Cd²⁺), weak (Ca²⁺), or low (Sr²⁺) velocities for ATP hydrolysis. The K_{M(app)} values for the M•ATP²⁻ substrates were not diagnostic of metal efficiency (V/K_{M}). The K_{M(app)} for Mg²⁺, Mn²⁺, and Co²⁺ were comparable; yet the efficiencies (Vₘₐₓ/K_{M} or Vₚₑₐₖ/K_{M}) for Mg²⁺ and Mn²⁺ were 1.5 and 1.6 times that of Co²⁺, respectively. Finally, inspection of the composite table showed only two possible periodic trends. These were for the series Mg²⁺, Ca²⁺, Sr²⁺ (group 2A) and Zn²⁺, Cd²⁺, Hg²⁺ (group 2B). The group 2A metals all showed Michaelis-Menten kinetics. Furthermore, a steady drop in the Vₘₐₓ and Vₘₐₓ/K_{M} was observed as the study progessed down the group. A similar drop in the Vₚₑₐₖ (Vₘₐₓ) and the Vₚₑₐₖ/K_{M} (Vₘₐₓ/K_{M}) was observed for the group 2B metals. The only exception in the group 2A metals was Be²⁺, which produced no rho poly(C)-dependent activity. This anomaly may be attributed to the formation of an ATP inhibitory transition state complex upon binding to ADP. A nonlinear regression analysis program (SigmaPlot2001) was used to determine the Hill coefficients, n, seen as the final column in Table 1. Mg²⁺, Ca²⁺, Co²⁺, Hg²⁺, Mn²⁺, Sr²⁺, and VO²⁺ showed a Hill coefficient of 1, while for Cd²⁺, Ni²⁺, and Zn²⁺ n ranged from 2.9 (Ni²⁺) to 5.5 (Zn²⁺). The sigmoidal curves for Zn²⁺, Cd²⁺, and Ni²⁺ may signify cooperative behavior; however, they may also suggest additional binding sites on either poly(C) or rho.

B. Divalent Metal Activation of Rho in the Presence of MgCl₂. To provide additional information concerning the roles of divalent metals in rho activation, the poly(C)-dependent assays were repeated in the presence of 10 mM MgCl₂ and varying concentrations (10, 20, 40, 80, 125, 160, 250, 320, 500, 640 μM) of the divalent metals. (Table 2). Be²⁺, Ca²⁺, Cd²⁺, Co²⁺, Hg²⁺, Ni²⁺, Sr²⁺, VO²⁺, and Zn²⁺ were chosen and Mn²⁺ was excluded from this study since MnCl₂ precipitated the poly(C)-rho complex.

Ni²⁺, Ca²⁺, Cd²⁺, Co²⁺, VO²⁺, and Sr²⁺ had negligible effects on ATP hydrolysis rate in the presence of MgCl₂. This finding indicated that at 10 mM MgCl₂, Mg²⁺ can effectively compete with these metals for the essential sites in rho necessary for ATP hydrolysis and that these metals do not adversely interact with rho, ATP, ADP, and poly(C). Included in this list was Sr²⁺, a metal previously identified as either a nonactivator or an inhibitor. The lack of inhibition by Sr²⁺ in the presence of MgCl₂ indicated that this metal did not inactivate rho under these conditions, and thus is a nonactivator.

Four divalent metal ions (Be²⁺, Cu²⁺, Hg²⁺, Zn²⁺) inhibited rho poly(C)-dependent ATP hydrolysis in the presence of MgCl₂ (10 mM) (Table 2). The extent and pattern of inhibition was metal dependent. Cu²⁺ caused a modest loss of activity (˜30%), while larger decreases in activities were noted for Hg²⁺ and Zn²⁺. At 4000 μM Hg²⁺ and Zn²⁺, rho inhibition was ˜50%. Nonhyperbolic inactivation curves were observed for Cu²⁺, Hg²⁺, and Zn²⁺, suggesting that rho inhibition proceeded, in part, by a nonselective process and ZnCl₂ inhibition was found to be dependent on the MgCl₂ concentration. The estimated I₅₀ values for ZnCl₂ were 190, 190, and 4000 μM in the presence of 0, 1, and 10 mM MgCl₂, respectively. Furthermore, the I₅₀ value for ZnCl₂ increased only slightly when the poly(C) concentration was increased 10-fold. These findings indicated that the Zn²⁺ inhibition process was partially prevented by excess Mg²⁺ and that Zn²⁺ inhibition did not occur by RNA complexation. Of the 10 metals tested with 10 mM MgCl₂, only BeCl₂ showed a clear inhibition curve with an I₅₀ value of 63 μM (Table 2). At 100 μM BeCl₂ an almost complete loss of activity was observed. Previous studies have shown that BeX_{y} (X═F, Cl; y=2, 3) can bind with ADP to generate an ATP transition state complex that resembles ATP and inhibits ATP-dependent processes. See, Kanfer et al., (1994) Phospholipase D activity of isolated rat brain plasma membranes, FEBS Lett. 337, 251-254; Toda et al., (1971) Effects of cations on the inhibition of K⁺-activated phosphatase by beryllium, J. Biochem. 69, 73-82; and Santacroce et al., (1966) Inhibition of alkaline phosphatase by beryllium chloride in a continuous cellular line of mouse embryonic liver cells, Boll.-Soc. Ital. Biol. Sper. 42, 1023-1026.

The foregoing is illustrative of the present invention, and is not to be construed as limiting thereof. The invention is defined by the following claims, with equivalents of the claims to be included therein.

1. A pharmaceutical composition comprising:
a divalent metal or metal oxide; and
a compound comprising at least one thiol group, or a pharmaceutically acceptable salt, solvate or prodrug thereof, wherein said divalent metal and said at least one thiol group form a metal:thiol complex, in association with a pharmaceutically acceptable carrier, excipient or diluent.

2. The pharmaceutical composition of claim 1, wherein said divalent metal or metal oxide is selected from the group consisting of Ba²⁺, Be²⁺, Ca²⁺, Cd²⁺, Co²⁺, Cu²⁺, Fe²⁺, Hg²⁺, Mn²⁺, Ni²⁺, Pd²⁺, Pt²⁺, Sn²⁺, Sr²⁺, VO²⁺, and Zn²⁺.

3. The pharmaceutical composition of claim 1, wherein said compound comprising at least one thiol group comprises a cyclic or acyclic carbon framework wherein said cyclic or acyclic carbon framework comprises electron-donating or electron-withdrawing groups.

4. The pharmaceutical composition of claim 3, wherein said cyclic or acyclic carbon framework is aliphatic, heterocyclic, aromatic, or heteroaromatic, and said cyclic or acyclic carbon framework comprises single, double or triple bonds.

5. The pharmaceutical composition of claim 1, wherein said compound is an optical isomer, enantiomer, diastereomer, racemate or stereochemical mixture thereof.

6. The pharmaceutical composition of claim 1, wherein said compound comprising at least one thiol group is selected from the group consisting of dithiothreitol, dithioerythritol, 1,4-dihydroxy-2,3-dimercaptobutane, 1,2-ethanedithiol, 3,4-dimercaptotoluene, trithiocyanuric acid, 2,5-dimercapto-1,3,4-thiadiazole, 2,3-dimercapto-1-propanol, 2-mercapto-3-butanol, β-mercaptoethanol, 2-mercaptoethylamine, 1-monothioglycerol, 2,3-butanedithiol, 1,4-butanedithiol, 1,2-propanedithil, 1,3-propanedithiol, benzene-1,2-dithiol, 1,2-benzenedimethanedithiol, 2,3-dimercaptopyridine, 3,4-dimercaptopyridine, 4-mercaptopyridine, 3-mercaptopyridine, 2-mercaptopyridine, 2-mercaptoethylsulfide, 2-mercaptoethyl ether, bis-(2-mercaptoethyl)amine, mercaptoacetic acid, thiosalicyclic acid, 2-mercaptopyridine-N-oxide and dimethyldithiocarbamic acid.

7. The pharmaceutical composition of claim 1, wherein the divalent metal to compound comprising at least one thiol group ratio is at least 1:1.

8. The pharmaceutical composition of claim 1, wherein the divalent metal to compound comprising at least one thiol group ratio is between 1:1 and 2:1.

9. A method for inhibiting a rho transcription termination factor, wherein the method comprises administering a chelating agent and a compound comprising at least one thiol group.

10. The method according to claim 9, wherein said chelating agent is a divalent metal or metal oxide.

11. The method according to claim 10, wherein said divalent metal or metal oxide is selected from the group consisting of Ba²⁺, Be²⁺, Ca²⁺, Cd²⁺, Co²⁺, Cu²⁺, Fe²⁺, Hg²⁺, Mn²⁺, Ni²⁺, Pd²⁺, Pt²⁺, Sn²⁺, Sr²⁺, VO²⁺, and Zn²⁺.

12. The method according to claim 9, wherein said compound comprising at least one thiol group comprises a cyclic or acyclic carbon framework wherein said cyclic or acyclic carbon framework comprises electron-donating or electron-withdrawing groups.

13. The method according to claim 12, wherein said cyclic or acyclic carbon framework is aliphatic, heterocyclic, aromatic, or heteroaromatic, and said cyclic or acyclic carbon framework comprises single, double or triple bonds.

14. The method according to claim 9, wherein said compound comprising at least one thiol group is selected from the group consisting of dithiothreitol, dithioerythritol, 1,4-dihydroxy-2,3-dimercaptobutane, 1,2-ethanedithiol, 3,4-dimercaptotoluene, trithiocyanuric acid, 2,5-dimercapto-1,3,4-thiadiazole, 2,3-dimercapto-1-propanol, 2-mercapto-3 -butanol, β-mercaptoethanol, 2-mercaptoethylamine, 1-monothioglycerol, 2,3-butanedithiol, 1,4-butanedithiol, 1,2-propanedithiol, 1,3-propanedithiol, benzene-1,2-dithiol, 1,2-benzenedimethanedithiol, 2,3-dimercaptopyridine, 3,4-dimercaptopyridine, 4-mercaptopyridine, 3-mercaptopyridine, 2-mercaptopyridine, 2-mercaptoethylsulfide, 2-mercaptoethyl ether, bis-(2-mercaptoethyl)amine, mercaptoacetic acid, thiosalicyclic acid, 2-mercaptopyridine-N-oxide and dimethyldithiocarbamic acid.

15. A method of treating bacteria or fungi comprising administering a therapeutically effective amount of a composition comprising a divalent metal or metal oxide and a compound comprising at least one thiol group.

16. The method according to claim 15, wherein said divalent metal or metal oxide is selected from the group consisting of Ba²⁺, Be²⁺, Ca²⁺, Cd²⁺, Co²⁺, Cu²⁺, Fe²⁺, Hg²⁺, Mn²⁺, Ni²⁺, Pd²⁺, Pt²⁺, Sn²⁺, Sr²⁺, VO²⁺, and Zn²⁺.

17. The method according to claim 15, wherein said compound comprising at least one thiol group comprises a cyclic or acyclic carbon framework wherein said cyclic or acyclic carbon framework comprises electron-donating or electron-withdrawing groups.

18. The method according to claim 15, wherein said cyclic or acyclic carbon framework is aliphatic, heterocyclic, aromatic, or heteroaromatic, and said cyclic or acyclic carbon framework comprises single, double or triple bonds.

19. The method according to claim 15, wherein said compound comprising at least one thiol group is selected from the group consisting of dithiothreitol, dithioerythritol, 1,4-dihydroxy-2,3-dimercaptobutane, 1,2-ethanedithiol, 3,4-dimercaptotoluene, trithiocyanuric acid, 2,5-dimercapto-1,3,4-thiadiazole, 2,3-dimercapto-1-propanol, 2-mercapto-3-butanol, β-mercaptoethanol, 2-mercaptoethylamine, 1-monothioglycerol, 2,3-butanedithiol, 1,4-butanedithiol, 1,2-propanedithiol, 1,3-propanedithiol, benzene-1,2-dithiol, 1,2-benzenedimethanedithiol, 2,3-dimercaptopyridine, 3,4-dimercaptopyridine, 4-mercaptopyridine, 3-mercaptopyridine, 2-mercaptopyridine, 2-mercaptoethylsulfide, 2-mercaptoethyl ether, bis-(2-mercaptoethyl)amine, mercaptoacetic acid, thiosalicyclic acid, 2-mercaptopyridine-N-oxide and dimethyldithiocarbamic acid.

20. The method according to claim 15, wherein said bacteria is gram-positive and/or gram-negative bacteria.

21-31. (canceled)

32. A pharmaceutical composition comprising:
a divalent metal selected from the group consisting of Cd²⁺, Ni²⁺ and Zn²⁺; and
a compound comprising at least one thiol group wherein said compound is selected from the group consisting of dithiothreitol, dithioerythritol and 2,3-dimercapto-1-propanol.

33. The pharmaceutical composition of claim 32, wherein the metal to compound ratio is between 1:1 and 4:1.

34. The pharmaceutical composition of claim 32, wherein the composition is in liquid, semi-solid or solid form.
